(19) United States
(12) Patent Application Publication
(10) Pub. No.: US 2011/0009463 A1
(43) Pub. Date: Jan. 13, 2011

(54) GERANYLGERANYL TRANSFERASE INHIBITORS AND METHODS OF MAKING AND USING THE SAME

(76) Inventors: Yuri Karl Petersson, Durham, NC (US); Xiang Simon Wang, Durham, NC (US); Patrick John Casey, Chapel Hill, NC (US); Alexander Tropsha, Chapel Hill, NC (US)

Correspondence Address: SHERIDAN ROSS PC, 1560 BROADWAY, SUITE 1200, DENVER, CO, 80202, US

(21) Appl. No.: 12/679,604

(22) PCT Filed: Oct. 17, 2008
(86) PCT No.: PCT/US08/11841

§ 371 Date: Aug. 26, 2010

Related U.S. Application Data
(60) Provisional application No. 60/980,594, filed on Oct. 17, 2007.

(51) Int. Cl. A61K 31/41; A61K 31/4196; A61K 31/4152; A61K 31/415; A61K 31/341; A61P 27/06; A61P 27/02; A61P 17/06; A61P 19/02; A61P 35/00; A61P 29/00; A61P 9/10; A61P 31/12; A61P 31/18; A61P 31/14; A61P 31/10; A61P 31/04; A61P 33/02; A61P 9/00; A61P 15/00; A61P 3/00

(52) U.S. Cl. 514/381; 514/384; 514/407; 514/406; 514/471

(57) ABSTRACT

The present invention is directed to compounds useful in the treatment of diseases associated with prenylation of proteins and pharmaceutically acceptable salts thereof, to pharmaceutical compositions comprising same, and to methods for inhibiting protein prenylation in an organism using the same.

### GOVERNMENT INTEREST

This invention was made with Government support under grant number F32-GM073420 awarded by the National Institutes of Health (NIH). The Government has certain rights in this invention.

### FIELD OF INVENTION

This present invention relates to novel compounds useful in the treatment of diseases affected by modulating the prenylation of proteins.

BACKGROUND OF INVENTION

The mammalian Ras proteins are a family of guanosine triphosphate (GTP) binding and hydrolyzing proteins that regulate cell growth and differentiation. Their overproduction or mutation can lead to uncontrolled cell growth, and has been implicated as a cause or aggravating factor in a variety of diseases including cancer, inflammation, multiple sclerosis, restenosis, psoriasis, endometriosis, atherosclerosis, viral or yeast infections including Hepatitis C and HIV, apoptosis, angiogenesis, rheumatoid arthritis, psoriasis, glaucoma, diabetic retinopathy and corneal neovascularization.

Ras proteins share characteristic C-terminal sequences termed the CAAX motif, wherein C is Cys, A is an amino acid, usually an aliphatic amino acid, and X is an aliphatic amino acid or other type of amino acid. The biological activity of the proteins is dependent upon the post-translational modification of these sequences by isoprenoid lipids. For proteins having a C-terminal CAAX sequence, this modification, which is called prenylation, occurs in at least three steps: the addition of either a 15 carbon (farnesyl) or 20 carbon (geranylgeranyl) isoprenoid to the Cys residue, the proteolytic cleavage of the last three amino acids from the C-terminus, and the methylation of the new C-terminal carboxylate (Zhang and Casey, Ann. Rev. Biochem. 1996, 65:241-269). The prenylation of some proteins may include a fourth step; the palmitoylation of one or two Cys residues N-terminal to the farnesylated Cys.

Ras-like proteins terminating with XXCC or XCXC motifs can also be prenylated and are modified by geranylgeranylation on the Cys residues. These proteins do not require an endoproteolytic processing step. While some mammalian cell proteins terminating in XCXC are carboxymethylated, it is not clear whether carboxymethylation follows prenylation of proteins terminating with XXCC motifs (Clarke, Ann. Rev. Biochem., 1992, 61:355-386). For all Ras-like proteins, however, addition of the isoprenoid is the first step of prenylation, and is required for the subsequent steps (Cox and Der, Critical Rev. Oncogenesis, 1992, 3:365-400; Ashby et al., Curr. Opinion Lipidology, 1998, 9:99-102).

Three enzymes have been found to catalyze protein prenylation: farnesyl-protein transferase (FPTase), geranylgeranyl-protein transferase type I (GGPTase-I), and geranylgeranyl-protein transferase type-II (GGPTase-II, also called Rab GGPTase). These enzymes are present in both yeast and mammalian cells. Schafer and Rine (Annu. Rev. Genet., 1992, 30:209-237). U.S. Pat. No. 5,578,477 discloses a method of purifying FPTase using recombinant technology and yeast host cells. Such techniques are useful in the elucidation of the enzyme structures.

FPTase and GGPTase-I are α/βheterodimeric enzymes that share a common α subunit; the β subunits are distinct but share approximately 30% amino acid identity (Brown and Goldstein, Nature, 1993, 366:14-15; Zhang et al., J. Biol. Chem., 1994, 269:3175-3180). GGPTase II has different α and β subunits, and complexes with a third component (REP, Rab Escort Protein) that presents the protein substrate to the α/β catalytic subunits. GGPTase proteins, and the nucleic acid sequence encoding them, are disclosed by U.S. Pat. No. 5,789,558 and WO 95/20651. U.S. Pat. No. 5,141,851 discloses the structure of a FPTase protein.

Each of these enzymes selectively uses farnesyl diphosphate or geranylgeranyl diphosphate as the isoprenoid donor, and selectively recognizes the protein substrate. FPTase farnesylates CAAX-containing proteins that end with Ser, Met, Cys, Gln or Ala. GGPTase-I geranylgeranylates CAAX-containing proteins that end with Leu or Phe. For FPTase and GGPTase-I, CAAX tetrapeptides comprise the minimum region required for interaction of the protein substrate with the enzyme. GGPTase-II modifies XXCC and XCXC proteins, but its interaction with protein substrates is more complex, requiring protein sequences in addition to the C-terminal amino acids for recognition. Enzymological characterization of FPTase, GGPTase-I and GGPTase-II has demonstrated that it is possible to selectively inhibit only one of these enzymes (Moores et al., J. Biol. Chem., 1991, 266:17438).

GGPTase-I transfers a geranylgeranyl group from the prenyl donor geranylgeranyl diphosphate to the cysteine residue of substrate CAAX protein (Clarke, Annu. Rev. Biochem., 1992, 61:355-386; Newman and Magee, Biochim. Biophys. Acta, 1993, 1155:79-96). Known targets of GGPTase-I include the gamma subunits of brain heterotrimeric G proteins and Ras-related small GTP-binding proteins such as RhoA, RhoB, RhoC, CDC42Hs, Rac1, Rac2, Rap1A and Rap1B. The proteins RhoA, RhoB, RhoC, Rac1, Rac2 and CDC42Hs have roles in the regulation of cell shape (Ridley and Hall, Cell, 1992, 70:389-399; Ridley et al., Cell, 1992, 70:401-410; Bokoch and Der, FASEB J. 1993, 7:750-759). Rac and Rap proteins play roles in neutrophil activation.

It has been found that the ability of Ras proteins to affect cell shape is dependant upon Rho and Rac protein function. (Mackey and Hall, J. Biol. Chem., 1998, 273:20688-20695). It thus follows that because Rho and Rac proteins require geranylgeranylation for function, an inhibitor of GGPTase-I will block the functions of these proteins, and may therefore be useful as, for example, an anti-cancer agent. For example, GGPTase-I inhibitors can arrest human tumor cells that lack p53 in G0/G1, and induce the accumulation of p21^{WAP}. This suggests that these inhibitors could be used to restore growth arrest in cells lacking functional p53 (Vogt et al., J. Biol. Chem., 1997, 272:27224-27229). Noteworthy in this regard are reports indicating that K-Ras, the form of Ras gene most associated with human cancers, can be modified by GGPTase-I in cells where FPTase is inhibited (Whyte et al., J. Biol. Chem., 1997, 272:14459-14464). Since geranylgeranylated Ras has been reported to be as efficient as the farnesylated form in cell transformation studies, K-Ras cancers could be treated with GGPTase-I inhibitors (Lerner et al., J. Biol. Chem., 1995, 270:26770-26773).

In addition to cancer, there are other pathological conditions for which GGPTase inhibitors may be used as intervention agents. These include, for example, the intimal hyperplasia associated with restenosis and atherosclerosis. Pulmonary artery smooth muscle cells seem particularly sensitive to inhibition of GGPTase-I, and treatment of such cells with a GGPTase inhibitor resulted in a superinduction of their inducible nitric-oxide synthase (NOS-2) by interleukin-1 □Finder et al., J. Biol. Chem., 1997, 272:13484-13488).

GGPTase inhibitors may also be used as anti-fungal agents. In S. cerevisiae and Candida albicans, and apparently most other fungi, cell wall biosynthesis is controlled by a Rho-type protein that is modified by the fungal GGPTase-I (Qadota et al., Science, 1996, 272:279-281). Selective inhibition of the fungal enzyme would diminish cell wall integrity, and thus be lethal to fungal cells.

Numerous other prenylation inhibitors have been studied. Some examples of these are disclosed by U.S. Pat. Nos. 5,420,245; 5,574,025; 5,523,430; 5,602,098; 5,631,401; 5,705,686; 5,238,922; 5,470,832; and 6,191,147; and by European Application Nos. 856,315 and 537,008. The effectiveness and specificity of these inhibitors vary widely, as do their chemical structures, and many of them are difficult to synthesize and purify.

Therefore, there is a need for new, more effective prenyl-protein transferase inhibitors.

SUMMARY OF INVENTION

The present invention provides a group of structurally-related compounds that are effective inhibitors of protein prenylation.

The present invention also provides a pharmaceutical composition comprising at least one compound of the present invention, or a pharmaceutically-acceptable salt thereof, and a pharmaceutically-acceptable excipient.

The present invention also provides a method for inhibiting protein prenylation comprising contacting an isoprenoid transferase with a compound of the present invention or a pharmaceutically-acceptable salt thereof. As used herein, an “isoprenoid transferase” refers to any enzyme capable of transferring an isoprenoid group, for example, farnesyl or geranylgeranyl, to a protein, e.g., Ras or Ras-like proteins. Such isoprenoid transferases include FPTase, GGPTase I and GGPTase II. Unless the context requires otherwise, the term “contacting” refers to providing conditions to bring the compound into proximity to an isoprenoid transferase to allow for inhibition of activity of the isoprenoid transferase. For example, contacting a compound of the present invention with an isoprenoid transferase can be accomplished by administering the compound to an organism, or by isolating cells, e.g., cells in bone marrow, and admixing the cells with the compound under conditions sufficient for the compound to diffuse into or be actively taken up by the cells, in vitro or ex vivo, into the cell interior. When ex vivo administration of the compound is used, for example, in treating leukemia, the treated cells can then be re-infused into the organism from which they were taken.

Such method for inhibiting protein prenylation can be used, for example, in prevention and/or treatment of a disease or condition in a plant or animal that is caused, aggravated or prolonged by Ras or Ras-like protein prenylation. In animals, such diseases include, but are not limited to, cancer, inflammation, multiple sclerosis, restenosis, psoriasis, endometriosis, atherosclerosis, ischemia, myocardial ischemic disorders such as myocardial infarction, high serum cholesterol levels, viral infections including Hepatitis C and HIV, fungal infections, yeast infections, bacterial and protozoan infections, apoptosis, angiogenesis, rheumatoid arthritis, psoriasis, glaucoma, macular degeneration, diabetic retinopathy and disorders related to abnormal angiogenesis including, but not limited to, corneal neovascularization. In plants, such diseases include yeast and viral infections.

### BRIEF DESCRIPTION OF DRAWINGS

FIG. 1 shows the results of an in vitro assay of the activity of compounds of the present invention for the inhibition of the GGPTase I enzyme.

FIG. 2 shows the results of an in vitro assay of the activity of compounds of the present invention for the inhibition of the FPTase I enzyme.

### DESCRIPTION OF EMBODIMENTS

As used herein, the term “organism” includes plants and animals. Exemplary animals include mammals, fish, birds, insects, and arachnids. Humans can be treated with the compounds of the invention and fall within the mammal sub-category.

As used herein, the term “CAAX” means a C-terminal peptide sequence wherein C is Cys, A is an amino acid, usually an aliphatic amino acid, and X is another amino acid, usually Leu or Phe.

As used herein, the term “CAAX protein” means a protein comprising a CAAX sequence. As used herein, the term “XXCC” means a C-terminal peptide sequence wherein C is Cys and X is another amino acid, usually Leu or Phe.

As used herein, the term “XXCC protein” means a protein comprising a XXCC sequence.

As used herein, the term “XCXC” means a C-terminal peptide sequence wherein C is Cys and X is another amino acid, usually Leu or Phe.

As used herein, the term “XCXC protein” means a protein comprising a XCXC sequence.

As used herein, the term “Ras or Ras-like protein” encompasses Ras proteins, brain heterotrimeric G proteins, and other GTP-binding proteins such as members of the Rho, Rac and Rab family including, but not limited to, RhoA, RhoB, RhoC, CDC42Hs, Rac1, Rac2, Rap1A and Rap1B. A Ras or Ras-like protein may be a CAAX, XXCC, or XCXC protein. The term “Ras or Ras-like protein” as used herein also encompasses Rheb, inositol-1,4,5,triphosphate-5-phosphatase, and cyclic nucleotide phosphodiesterase and isoforms thereof, including nuclear lamin A and B, fungal mating factors, and several proteins in visual signal transduction.

As used herein, the term “Ras or Ras-like protein prenylation” means the prenylation of a Ras or Ras-like protein that is catalyzed or caused by GGPTase I, GGPTase II, or FPTase.

As used herein, the term “prenylation inhibitor” means a compound or mixture of compounds that inhibits, restrains, retards, blocks or otherwise affects protein prenylation, preferably Ras or Ras-like protein prenylation. A prenylation inhibitor may inhibit, restrain, retard, or otherwise affect the activity of GGPTase I, GGPTase II, and/or FPTase.

As used herein, the term “a pharmaceutically-acceptable salt thereof' refers to salts prepared from pharmaceutically-acceptable nontoxic acids or bases including inorganic acids and bases and organic acids and bases. Examples of such inorganic acids are hydrochloric, hydrobromic, hydriodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, tartaric, citric and galacturonic. Examples of suitable inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), lysine and procaine. Preferred salts of the compounds of this invention are TFA and acetate salts.

The phrase “therapeutically effective amount of prenylation inhibitor” as used herein means that amount of prenylation inhibitor which alone or in combination with other drugs provides a therapeutic benefit in the treatment, management, or prevention of conditions in a plant or animal that are caused, aggravated or prolonged by Ras or Ras-like protein prenylation. Such conditions include, but are not limited to, cancer, restenosis, psoriasis, endometriosis, atherosclerosis, ischemia, myocardial ischemic disorders such as myocardial infarction, high serum cholesterol levels, viral infection, fungal infections, yeast infections, bacteria and protozoa infections, conditions related to abnormal bone resorption, undesired angiogenesis, abnormal angiogenesis or abnormal proliferation such as, but not limited to, corneal neovascularization.

“Alkyl” groups according to the present invention are aliphatic hydrocarbons which can be straight, branched or cyclic. Alkyl groups optionally can be substituted with one or more substituents, such as a halogen, alkenyl, alkynyl, aryl, hydroxy, amino, thio, alkoxy, carboxy, oxo or cycloalkyl. There may be optionally inserted along the alkyl group one or more oxygen, sulfur or substituted or unsubstituted nitrogen atoms. Exemplary alkyl groups include methyl, ethyl, propyl, i-propyl, n-butyl, t-butyl, bicycloheptane (norbornane), cyclobutane, dimethyl-cyclobutane, cyclopentane, cyclohexane, fluoromethyl, difluoromethyl, trifluoromethyl, chloromethyl, trichloromethyl, and pentafluoroethyl. Preferably, alkyl groups have from about 1 to about 20 carbon atom chains, more preferably from about 1 to about 10 carbon atoms, still more preferably from about 1 to about 6 carbon atoms, and most preferably from about 1 to about 4 carbon atoms.

“Aryl” groups are monocyclic or bicyclic carbocyclic or heterocyclic aromatic ring moieties. Aryl groups can be substituted with one or more substituents, such as a halogen, alkenyl, alkyl, alkynyl, hydroxy, amino, thio, alkoxy or cycloalkyl.

“Heteroaryl” refers to monocyclic or bicyclic aromatic ring having at least one heteroatom selected from nitrogen, sulfur, phosphorus and oxygen. Preferred heteroaryls are 5- and 6-membered aromatic rings which contain from about 1 to about 3 heteroatoms. Examples of heteroaryl groups include, but are not limited to, pyridinyl, imidazolyl, pyrimidinyl, pyrazolyl, (1,2,3)- and (1,2,4)-triazolyl, pyrazinyl, tetrazolyl, furyl, thienyl, isoxazolyl, oxazolyl, pyrrolyl, thiazolyl, pyrrole, thiophenyl, furanyl, pyridazinyl, isothiazolyl, and S-triazinyl.

“N-heteroaryl” refers to monocyclic or bicyclic aromatic ring having at least one nitrogen atom in the aromatic ring moiety. Exemplary N-heteroaryls include, but are not limited to, pyridinyl, imidazolyl, pyrimidinyl, pyrazolyl, (1,2,3)- and (1,2,4)-triazolyl, pyrazinyl, tetrazolyl, isoxazolyl, oxazolyl, pyrrolyl, pyrrole, pyridazinyl, and isothiazolyl. Preferably, N-heteroaryl is pyridinyl. More preferably, N-heteroaryl is pyridin-3-yl.

The term “aryl containing at least one nitrogen substituent” refers to an aryl moiety having a substituent such as an amino, including mono-, di-, and tri-alkyl amino groups; amido; or C₁-C₄ alkyl groups having an amino or an amido substituent. Preferably, an “aryl containing at least one nitrogen substituent” is an aryl moiety having amino, amido or C₁-C₂ alkyl having an amino or amido substituent; more preferably amino, amido or C₁ alkyl having an amino or amido substituent; still more preferably an amino or amido substituent; and most preferably an amino substituent.

The term “peptoids” or “polypeptoids” refers to poly-(N-substituted glycine) chains. These peptidomimetic molecules have a number of particular advantages as discussed below. For example, peptoids are synthetic and non-natural polymers with controlled sequences and lengths, that may be made by automated solid-phase organic synthesis to include a wide variety of side-chains having different chemical functions. Peptoids have a number of notable structural features in comparison to peptides. For example, peptoids lack amide protons; thus, no intrachain hydrogen-bond network along the polymer backbone is possible, unless hydrogen-bond donating side-chains are put in the peptoid chain. In addition, whereas the side-chain (“R”) groups on biosynthetically produced peptides must be chosen from among the 20 amino acids, peptoids can include a wide variety of different, non-natural side-chains because in peptoid synthesis the R group can be introduced as a primary amine. This is in contrast to synthetic peptides for which the incorporation of non-natural side-chains requires the use of non-natural a-protected amino acids. Polypeptoid (or peptoids) can be synthesized in a sequence-specific fashion using an automated solid-phase protocol, e.g., the sub-monomer synthetic route. See, for example, Wallace et al., Adv. Amino Acid Mimetics Peptidomimetics, 1999, 2, 1-51 and references cited therein, all of which are incorporated herein in their entirety by this reference.

The flexibility of sub-monomer synthesis allows attachment of side-chains that satisfy the requirements of specific needs, e.g., hydrophilicity or hydrophobicity. Another advantage of the peptoid synthetic protocol is that it allows easy production of peptoid-peptide chimerae. In a single automated solid-phase protocol, one can alternate the addition of peptoid monomers with the addition of □-Fmoc-protected peptide monomers, the latter added by standard Fmoc coupling protocols employing activating agents such as pyBrop or pyBop (i.e., 1H-benzotriazol-1-yloxy-tris(pyrrolidino)phosphonium hexafluorophosphate).

Unless otherwise stated, the term “aromatic group” refers to aryl and heteroaryl groups.

The terms “substituted,” “substituted derivative” and “derivative” when used to describe a chemical moiety means that at least one hydrogen bound to the unsubstituted chemical moiety is replaced with a different atom or a chemical moiety. Examples of substituents include, but are not limited to, alkyl, halogen, nitro, cyano, heterocycle, aryl, heteroaryl, amino, amide, hydroxy, ester, ether, carboxylic acid, thiol, thioester, thioether, sulfoxide, sulfone, carbamate, peptidyl, PO₃H₂, and mixtures thereof.

The term “cancer” encompasses, but is not limited to, myeloid leukemia; malignant lymphoma; lymphocytic leukemia; myeloproliferative diseases; solid tumors including benign tumors, adenocarcinomas, and sarcomas; and blood-borne tumors. The term “cancer” as used herein includes, but is not limited to, cancers of the cervix, breast, bladder, colon, stomach, prostate, larynx, endometrium, ovary, oral cavity, kidney, testis and lung.

The terms “compound of the present invention,” “compound of this invention,” “compound of the invention,” “prenylation inhibitor of the present invention,” “prenylation inhibitor of this invention,” and “prenylation inhibitor of the invention” are used interchangeably to refer to the compounds and complexes disclosed herein, and to their pharmaceutically acceptable salts, solvates, hydrates, polymorphs, and clatherates thereof, and to crystalline and non-crystalline forms thereof.

The term “excipient” means a usually inert substance (as gum arabic or starch) that forms a vehicle or part of a vehicle for a pharmaceutical form, such as a unit dosage formulation.

The present invention is based on compounds that are potent prenylation inhibitors. These compounds inhibit the activity of one or more of the following: GGPTase I, GGPTase II, and FPTase. In particular embodiments, compounds of the present invention are selective inhibitors of GGPTase activity. In one particular embodiment, the compounds of the present invention, under the assay conditions disclosed in the Examples section, have an IC₅₀ value for GGPTase I of about 25 μM or less, more preferably about 10 μM or less, more preferably about 5 μM or less, more preferably about 10 nanomolar or less and most preferably between about 1 nanomolar and about 2 nanomolar.

This invention is further based upon the recognition that protein prenylation, in particular prenylation of CAAX, XXCC and/or XCXC proteins, is associated with a variety of diseases and/or conditions in plants and animals. In animals, such diseases include, but are not limited to, cancer, restenosis, psoriasis, endometriosis, atherosclerosis, ischemia, myocardial ischemic disorders such as myocardial infarction, high serum cholesterol levels, viral infection, fungal infections, yeast infections, bacteria and protozoa infections, proliferative disorders, and disorders related to abnormal angiogenesis including, but not limited to, corneal neovascularization. In plants, such diseases include yeast and viral infections.

In one particularly preferred embodiment, compounds of the invention are administered in the treatment of glaucoma. In this embodiment, one or more of the compounds of the invention may be administered topically to the eye, preferably in the form of ophthalmic drops or ophthalmic ointments, or taken orally or by injection. It will be understood that a treating health care provider will determine the correct dosage of the compound to administer considering such factors as the state of the patient's glaucoma, the patient's prognosis, the route of administration and the patient's physical characteristics.

In a related embodiment, the compounds of the invention are administered in the treatment of macular degeneration. As described above with respect to the treatment of glaucoma, one or more of the compounds of the invention may be administered topically to the eye, preferably in the form of ophthalmic drops or ophthalmic ointments, or taken orally or by injection for the treatment of macular degeneration, at a dosage and frequency best determined by the treating health care provider.

Another embodiment of the invention is the topical application of compounds of the present invention for the prevention or treatment of skin pathologies associated with prenylation including psoriasis and arthritis. In these instances, one or more compounds of the invention are administered topically to the patient under the direction of a health care provider to alleviate, treat or prevent the recurrence of psoriasis and arthritic conditions.

Another embodiment of the invention is a method of treating infectious diseases by the administration of at least one compound of the present invention to a patient infected with Hepatitis C, HIV or a yeast infection. In the treatment of these infectious conditions, the compounds of the invention may be administered orally or intravenously and may have significant effects following topical application in certain infectious conditions as well.

Compounds of the present invention useful for inhibition of protein prenylation are shown below. It should be recognized that reference to a compound, identification of a general chemical structure or a specific compound below and in the claims refers to the compound itself, as well as pharmaceutically acceptable salts thereof. Moreover, to the extent the structure of elements in the definitions of the R groups permits more than one site of attachment to the main structure, preferred sites of attachment for such elements are shown below in the exemplary specific structures an/or are dictated by the various methods of synthesis shown below.

One embodiment of the present invention provides compounds that may be used for inhibiting protein prenylation having the general structure of Formula I:

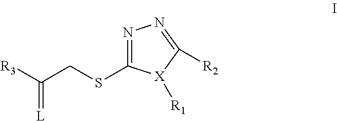

or a pharmaceutically-acceptable salt thereof,
wherein,

X is independently C or N;

L is independently O or CH₂;

R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;

R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-isopropylphenyl, 4-methylphenyl; and,

R₃ is H, NH₂, C(O)N(CH₃)₂, CO₂H, CN, CH₂OH, C(O)NH₂, CSNH₂, C(O)NH C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, N-(2,5-dichlorophenyl)amide, 3-chloro-N-methylaniline,

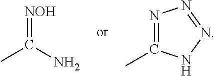

Another embodiment of the invention provides additional compounds that may be used for inhibiting protein prenylation having the general structure of Formula II:

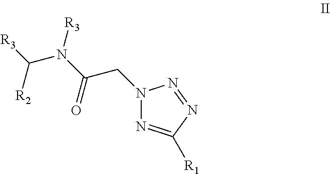

or a pharmaceutically-acceptable salt thereof,

wherein,

R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;

R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-methylphenyl, or 4-isopropylphenyl; and, methylthiadiazole, Each R₃ is independently H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-am inobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

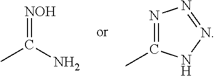

Another embodiment of the invention provides additional compounds that may be used for inhibiting protein prenylation having the general structure of Formula III:

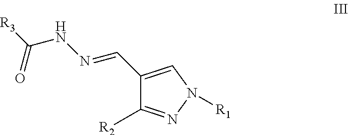

or a pharmaceutically-acceptable salt thereof,

wherein,

R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;

R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-methylphenyl, or 4-isopropylphenyl; and,

R₃ is H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

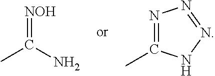

Another embodiment of the invention provides additional compounds that may be used for inhibiting protein prenylation having the general structure of Formula IV:

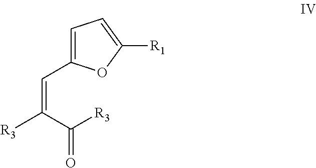

or a pharmaceutically-acceptable salt thereof,

wherein,

R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl; and,

Each R₃ is independently H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

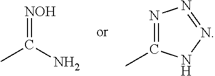

Particularly preferred compounds of the present invention useful for protein prenylation are shown in Table 1.

TABLE 1
Particularly preferred prenylation inhibitors of the invention

| | |
|---|---|
| En1 | 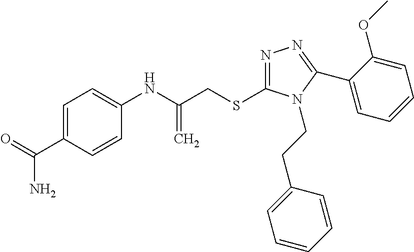 |
| En2 | 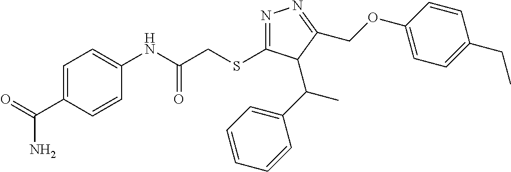 |
| As1 | 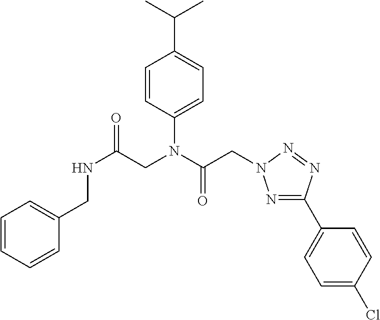 |
| As2 | 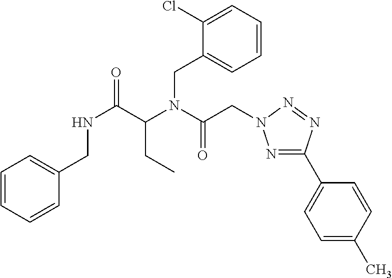 |
| Sig 1 | 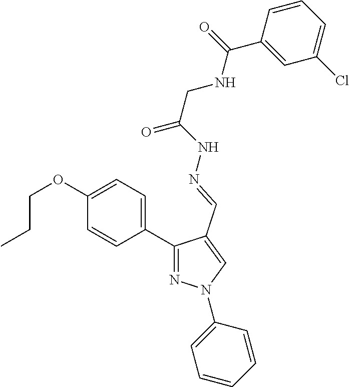 |
| Sig2 | 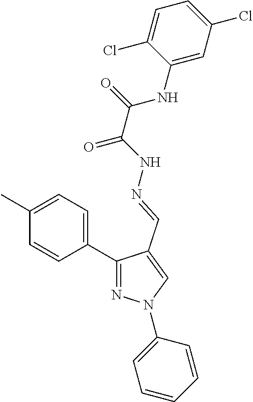 |
| Sig3 | 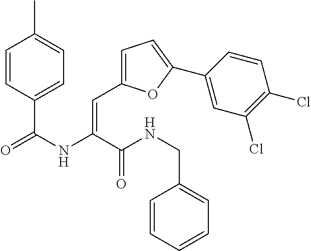 |

The compounds of the present invention can be synthesized from readily available starting materials. Various substituents on the compounds of the present invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in Protective Groups in Organic Synthesis, 2nd edition, T. H. Greene and P. G. M. Wuts, John Wiley & Sons, New York, N.Y., 1991, which is incorporated herein in its entirety by this reference. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

Since the compounds of the present invention can have certain substituents that are necessarily present, the introduction of each substituent is, of course, dependent upon the specific substituents involved and the chemistry necessary for their formation. Thus, consideration of how one substituent would be affected by a chemical reaction when forming a second substituent would involve techniques familiar to one of ordinary skill in the art. This would further be dependent on the ring involved.

It will be appreciated by those skilled in the art that compounds of the invention having a chiral center may exist in and be isolated in optically active and racemic forms. It is to be understood that the present invention encompasses any racemic, optically-active, regioisomeric or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine prenylation inhibitor activity using the standard tests described herein, or using other similar tests which are well known in the art. It is also to be understood that the scope of this invention encompasses not only the various isomers that may exist but also the various mixtures of isomers that may be formed. For example, if the compound of the present invention contains one or more chiral centers, the compound can be synthesized enantioselectively or a mixture of enantiomers and/or diastereomers can be prepared and separated. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the compounds is based on the differences in the physical properties of diastereomers by attachment, either chemically or enzymatically, of an enantiomerically pure moiety resulting in forms that are separable by fractional crystallization, distillation or chromatography.

When the compound of the present invention contains an olefin moiety and such olefin moiety can be either cis- or trans-configuration, the compound can be synthesized to produce cis- or trans-olefin, selectively, as the predominant product. Alternatively, the compound containing an olefin moiety can be produced as a mixture of cis- and trans-olefins and separated using known procedures, for example, by chromatography as described in W. K. Chan, et al., J. Am. Chem. Soc., 1974, 96, 3642, which is incorporated herein by this reference.

The compounds of the present invention form salts with acids when a basic amino function is present and salts with bases when an acid function, e.g., carboxylic acid or phosphonic acid, is present. All such salts are useful in the isolation and/or purification of the new products. Of particular value are the pharmaceutically acceptable salts with both acids and bases. Suitable acids and bases are described above. In addition, hydrated, solvated and/or anhydrous forms of compounds disclosed herein are also encompassed in the present invention.

The compounds of present invention may be prepared by both conventional and solid phase synthetic techniques known to those skilled in the art. Useful conventional techniques include those disclosed by U.S. Pat. Nos. 5,569,769 and 5,242,940, and PCT publication No. WO 96/37476, each of which are incorporated herein in their entirety by this reference.

Combinatorial synthetic techniques, however, are particularly useful for the synthesis of the compounds of the present invention. See, e.g., Brown, Contemporary Organic Synthesis, 1997, 216; Felder and Poppinger, Adv. Drug Res., 1997, 30, 111; Balkenhohl et al., Angew. Chem. Int. Ed. Engl., 1996, 35, 2288; Hermkens et al., Tetrahedron, 1996, 52, 4527; Hermkens et al., Tetrahedron, 1997, 53, 5643; Thompson et al., Chem. Rev., 1996, 96, 555; and Nefzi et al., Chem. Rev., 1997, 2, 449-472.

The compounds of the present invention that are basic in nature are capable of forming a wide variety of different salts with various inorganic and organic acids. Although such salts must be pharmaceutically acceptable in order to be administered to organisms, it may be desirable to initially isolate compounds of the present invention from reaction mixtures as pharmaceutically unacceptable salts, which are then converted back to the free base compounds by treatment with an alkaline reagent, and subsequently converted to pharmaceutically acceptable acid addition salts. The acid addition salts of the basic compounds of this invention are readily prepared by treating the compounds with substantially equivalent amounts of chosen mineral or organic acids in aqueous solvent mediums, or in suitable organic solvents such as methanol and ethanol. Upon careful evaporation of these solvents, the desired solid salts are readily obtained. Desired salts can also be precipitated from solutions of the free base compounds in organic solvents by adding to the solutions appropriate mineral or organic acids.

Those compounds of the present invention that are acidic in nature are similarly capable of forming base salts with various cations. As above, when a pharmaceutically acceptable salt is required, it may be desirable to initially isolate a compound of the present invention from a reaction mixture as a pharmaceutically unacceptable salt, which can then be converted to a pharmaceutically acceptable salt in a process analogous to that described above. Examples of base salts include alkali metal or alkaline-earth metal salts and particularly sodium, amine and potassium salts. These salts are all prepared by conventional techniques. The chemical bases used to prepare the pharmaceutically acceptable base salts of this invention are those which form non-toxic base salts with the acidic compounds of the present invention. Such non-toxic base salts include those derived from pharmacologically acceptable cations such as sodium, potassium, calcium, magnesium, and various amine cations. These salts can easily be prepared by treating the corresponding acidic compounds with an aqueous solution containing the desired pharmacologically acceptable bases and then evaporating the resulting solution to dryness, preferably under reduced pressure. They may also be prepared by mixing lower alkanolic solutions to dryness in the same manner as before. In either case, stoichiometric quantities of reagents are preferably employed in order to ensure completeness of reaction and maximum yields of the desired final product.

This invention encompasses both crystalline and non-crystalline (e.g., amorphous) forms of the salts of the compounds of this invention. These salts can be used to increase the solubility or stability of the compounds disclosed herein. They may also aid in the isolation and purification of the compounds.

Suitable methods of synthesizing the compound of the present invention may yield mixtures of regioisomers and/or diastereomers. These mixtures, which are encompassed by the compounds and methods of the present invention, can be separated by any means known to those skilled in the art. Suitable techniques include high performance liquid chromatography (HPLC) and the formation and crystallization of chiral salts. See, e.g., Jacques et al., Enantiomers, Racemates and Resolutions, Wiley-Interscience, New York, N.Y., 1981; Wilen et al., Tetrahedron, 1977, 33, 2725; Eliel, Stereochemistry of Carbon Compounds, McGraw-Hill, New York, N.Y., 1962; and Wilen, Tables of Resolving Agents and Optical Resolutions, Eliel, ed., Univ. of Notre Dame Press, Notre Dame, Ind., 1972, p. 268. The resulting enantiomerically enriched compounds are encompassed by the present invention.

The ability of the compounds of the present invention to inhibit protein prenylation of, for example, Ras or Ras-like proteins, may be determined by methods known to those skilled in the art such as the methods shown in the Examples below, and by methods disclosed in the references incorporated herein. Compounds of the present invention are inhibitory in GGPTase I assays. For example, compounds of the present invention, at a concentration of 10 μM in the GGPTase I assay described in Example 4, preferably show a percent inhibition of at least about 20%, more preferably at least about 35% and more preferably at least about 50%. GGPTase I may be prepared and purified according to the method described by Zhang et al., J. Biol. Chem., 1994, 9, 23465-23470, and U.S. Pat. No. 5,789,558, which is incorporated herein in its entirety by this reference. GGPTase II may be prepared by a method as disclosed in, for example, Johannes et al., Eur. J. Biochem., 1996, 239, 362-368; and Witter and Poulter, Biochemistry, 1996, 35, 10454-10463, all of which are incorporated herein in their entirety by this reference. FPTase may be prepared and purified by methods such as those disclosed by U.S. Pat. Nos. 5,141,851 and 5,578,477, both of which are incorporated herein in their entirety by this reference.

The compounds of the present invention can be used for inhibiting protein prenylation by contacting an isoprenoid transferase with the compound. The compound can be contacted with a cell, in vitro or ex vivo, and be taken up by the cell. The compounds of the present invention can also be administered to an organism to achieve a desired effect. An organism may be a plant or an animal, preferably a mammal, and more preferably a human.

For inhibiting protein prenylation in an animal, the compound of the present invention can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

In one particular embodiment of the present invention, protein prenylation inhibition is administered to an organism to treat or prevent conditions in an organism that arise due to Ras or Ras-like protein prenylation. In animals, such diseases include, but are not limited to, cancer, inflammation, multiple sclerosis, restenosis, psoriasis, endometriosis, atherosclerosis, ischemia, myocardial ischemic disorders such as myocardial infarction, high serum cholesterol levels, viral infections including Hepatitis C and HIV, fungal infections, yeast infections, bacterial and protozoan infections, apoptosis, angiogenesis, rheumatoid arthritis, psoriasis, glaucoma, macular degeneration, diabetic retinopathy and disorders related to abnormal angiogenesis including, but not limited to, corneal neovascularization. In plants, such diseases include yeast and viral infections.

The method of the present invention can also include the administration of a dosage form comprising at least one compound of the present invention alone or in combination with other drugs or compounds. Other drugs or compounds that may be administered in combination with the compounds of the present invention may aid in the treatment of the disease or disorder being treated, or may reduce or mitigate unwanted side-effects that may result from the administration of the compounds.

The magnitude of a prophylactic or therapeutic dose of a compound of the present invention used in the prevention, treatment, or management of a disorder or condition can be readily determined by one of skill in the art using in vitro and in vivo assays such as those described below. As those of skill in the art will readily recognize, however, the magnitude of a prophylactic or therapeutic dose of a prenylation inhibitor will vary with the severity of the disorder or condition to be treated, the route of administration, and the specific compound used. The dose, and perhaps the dose frequency, will also vary according to the age, body weight, and response of the individual patient. Typically, a physician will determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/kg of body weight per day and preferably from about 0.1 to about 20 mg/kg of body weight per day and can be administered in several different dosage units. Higher dosages, on the order of about 2× to about 4×, may be required for oral administration. In another aspect, the therapeutic dosage can be sufficient to achieve blood levels of the therapeutic agent of between about 5 micromolar and about 10 micromolar.

In one exemplary application, a suitable amount of a compound of the present invention is administered to a mammal undergoing treatment for an eye disorder such as glaucoma or macular degeneration. Administration occurs in an amount of between about 0.1 mg/kg body weight to about 20 mg/kg body weight per day, preferably between about 0.5 mg/kg body weight to about 10 mg/kg body weight per day.

In another exemplary application, a suitable amount of a compound of this invention is administered to a mammal undergoing treatment for psoriasis or arthritis. The magnitude of a prophylactic or therapeutic dose of the compound will vary with the nature and severity of the condition to be treated, and with the particular compound and its route of administration. In general, however, administration of a compound of the present invention for treatment of these immune disorders occurs in an amount of between about 0.1 mg/kg body weight to about 100 mg/kg of body weight per day, preferably between about 0.5 mg/kg body weight to about 10 mg/kg of body weight per day.

It is recommended that children and patients aged over 65 years initially receive low doses, and that they then be titrated based on individual response(s) or blood level(s). It may be necessary to use dosages outside the ranges identified above in some cases as will be apparent to those skilled in the art. Further, it is noted that the clinician or treating physician will evaluate how and when to adjust, interrupt, or terminate therapy in conjunction with individual patient response.

When used to inhibit protein prenylation in plants, the compounds of the present invention may be administered as aerosols using conventional spraying techniques, or may be mixed or dissolved in the food, soil and/or water provided to the plants. Other methods of administration known in the art are also encompassed by the invention.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least about 0.1% of active compound. The percentage of the compositions and preparation can, of course, be varied and can conveniently be between about 1% to about 10% of the weight of the unit. The amount of active compound in such therapeutically useful compositions is such that a suitable dosage will be obtained. Preferred compositions or preparations according to the present invention are prepared such that an oral dosage unit form contains from about 1 to about 1000 mg of active compound.

The tablets, troches, pills, capsules and the like can also contain the following: binder(s) such as gum tragacanth, acacia, corn starch or gelatin; excipient(s) such as dicalcium phosphate; disintegrating agent(s) such as corn starch, potato starch, alginic acid and the like; lubricant(s) such as magnesium stearate; and sweetening agent(s) such as sucrose, lactose or saccharin can be added or a flavoring agent such as peppermint, oil of wintergreen, or cherry flavoring. When the unit dosage form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed.

The active compound can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid such that it is possible to be delivered by syringe. It can be stable under the conditions of manufacture and storage and must be preserved against the contaminating action of microorganisms such as bacteria and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions may be accomplished by the inclusion of agents delaying absorption in the injectable preparation, e.g., aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

Dosage forms for the topical or transdermal administration of compounds of this invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Because of their ease of administration, tablets and capsules represent the most advantageous oral unit dosage form, in which case solid pharmaceutical carriers are employed. If desired, tablets may be coated by standard aqueous or nonaqueous techniques.

In addition to the common dosage forms set out above, the compounds of the present invention may also be administered by controlled release means and/or delivery devices capable of releasing the active ingredient (prenylation inhibitor) at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. Examples of controlled release pharmaceutical compositions and delivery devices that may be adapted for the administration of the active ingredients of the present invention are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; and 5,733,566, the disclosures of which are incorporated herein in their entirety by this reference.

Pharmaceutical compositions for use in the methods of the present invention may be prepared by any methods known in the pharmaceutical sciences. Such methods are well known to the art and as described, for example, in Remington: The Science and Practice of Pharmacy, Lippincott, Williams & Wilkins, pubs, 20th edition (2000). All of these methods include the step of bringing the active ingredient into association with the carrier that constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

Additional objects, advantages, and novel features of this invention will become apparent to those skilled in the art upon examination of the following examples thereof, which are not intended to be limiting.

### Examples

### Example 1

This example illustrates a method for preparing and purifying GGPTase I.

GGPTase I was prepared and purified according to the method described by Zhang et al., J. Biol. Chem., 1994, 9, 23465-23470, which is incorporated herein in its entirety by this reference.

### Production of Recombinant Virus

Sf9 cells were obtained from the American Tissue Culture Collection. The cells were maintained in Grace's medium (GIBCO™), supplemented with about 3.3 mg/ml lactalbumin hydrolystate (DIFCO™), about 3.3 mg/ml yeastolate (DIFCO™), about 10% (v/v) fetal bovine serum (HYCLONE™ Laboratories), antibiotic-antimycotic mixture (GIBCO™), and about 0.1% Pluronic F-68 (GIBCO™) in 125 ml Spinner flask (available from Techne, Princeton, N.J.). To generate recombinant baculovirus, about 2×10⁶ Sf9 cells were transfected with about 0.5 μg of BACULOGOLD™ wild-type viral DNA (available from PHARMINGEN™) and about 2 μg of either pVL-Fα (for α subunit expression) or pVL-Gβ(for GGPTase-Iβ subunit expression) using calcium-phosphate precipitation according to the manufacturer's instructions. The virus from each transfection was harvested after about 4 days and screened using a plaque assay as described by Summers and Smith, A Manual of Methods for Baculovirus Vectors and Insect Cell Culture Procedures, Texas Agricultural Experimentation Station, Bulletin #1555 (1987). Recombinant viruses obtained from this screen were subjected to two further rounds of plaque amplification to obtain purified viruses.

### Production and Purification of Recombinant GGPTase-I

The purified recombinant viruses containing the cDNA sequences for the α subunit of FPTase and GGPTase, and the β subunit of GGPTase-I were used to co-infect about 1.5×10⁶ Sf9 cells at multiplicities of infection of 5. Cells were harvested at about 65 hours post-infection by centrifugation at about 800×g for about 15 minutes. The cells were washed once with phosphate-buffered saline and the resulting cell pellet flash-frozen in liquid nitrogen. Cell extracts were prepared by thawing the cell suspension in 5 volumes of about 20 mM Tris-HCl, pH 7.7, about 1 mM EDTA, 1 mM EGTA, about 1 mM and a protease inhibitor mixture (Moomaw et al., Methods Enzymol., 1995, 250, 12-21), incubating the cell suspension on ice for about one hour, and disrupting using six strokes of a Dounce homogenizer. The resulting extract was centrifuged for about 1 hour at about 30,000×g, and the supernatant (designated as the soluble extract) was fractionated on a 5.0×10.0 cm column of DEAE-Sephacel (available from Pharmacia). The DEAE-Sephacel was first equilibrated with 50 mM Tris-Cl, pH 7.7, 1 mM DTT (Buffer A) at 4° C. The soluble extract containing about 160 mg protein was loaded into the DEAE column, which was then washed with about 50 ml Buffer A and eluted with a 200 ml gradient of 0-500 mM NaCl in Buffer A. Fractions of 3 ml were collected. The fractions containing the peak of GGPTase-I activity were pooled, concentrated and exchanged into Buffer A, and then loaded into a Q-HP column (1.0×20 cm, available from Pharmacia). The column was washed with about 20 ml of buffer A and eluted with a 200 ml gradient of 0-500 mM NaCl in Buffer A. The peak fractions, containing essentially homogeneous GGPTase-I, were pooled, flash-frozen in aliquots and stored at −80° C.

### Example 2

This example illustrates a method for determining GGPTase-I activity.

GGPTase-I activity was determined by the method of Casey et al., Proc. Natl. Acad. Sci. USA, 1991, 88, 8631-8635. This method measures the transfer of isoprenoid from ³H-geranylgeranyl diphosphate (GGPP) into a Ras protein with a C-terminal leucine-for-serine substitution (designated as Ras-CVLL).

### Example 3

This example illustrates GGPTase I and FPTase inhibitory activities of some of the compounds of the present invention.

Assays for the inhibition studies of GGPTase I were performed in a manner analogous to that described by Casey, et al., Proc. Natl. Acad. Sci. USA, 1991, 88, 8631-8635, with the following modifications. For those assays, the reaction mixtures contained the following components in 50 μl: 0.25 μM [³H]GGPP (specific activity 8-10 Ci/mmol), 2.5 μM Ras-CVLL, 50 mM Tris-Cl, pH 7.7, 20 mM KCl, 5 mM MgCl₂, 5 μM ZnCl₂, 1 mM DTT, 0.5 mM Zwittergent 3-14 and the desired amount of the compound to be tested for inhibitory potential. After pre-equilibrating the assay mixture at 30° C. in the absence of the enzyme, the reaction was initiated by addition of the enzyme (75 ng). Following an about 10 minute incubation at about 30° C., the reactions were terminated by addition of about 0.5 ml of about 4% SDS. About 40 mg of bovine brain membranes was added to the samples to enhance recovery during precipitation. Product was precipitated by addition of about 0.5 ml of 30% TCA, allowed to stand at room temperature for about 15 minutes, and processed by filtration through glass-fiber filters as described previously (Reiss et al., Methods: Companion to Methods in Enzymology, 1991, 1, 241-245). Reactions were never allowed to proceed to more than 10% completion based on the limiting substrate. Assays for the inhibition studies of FPTase were performed analogous the GGPTase I inhibition studies, except [³H]GGPP was replaced with 0.25 μM of [³H]FPP (specific activity 8-10 ci/mmol) and Ras-CVLL was replaced with 1 μM H-Ras.

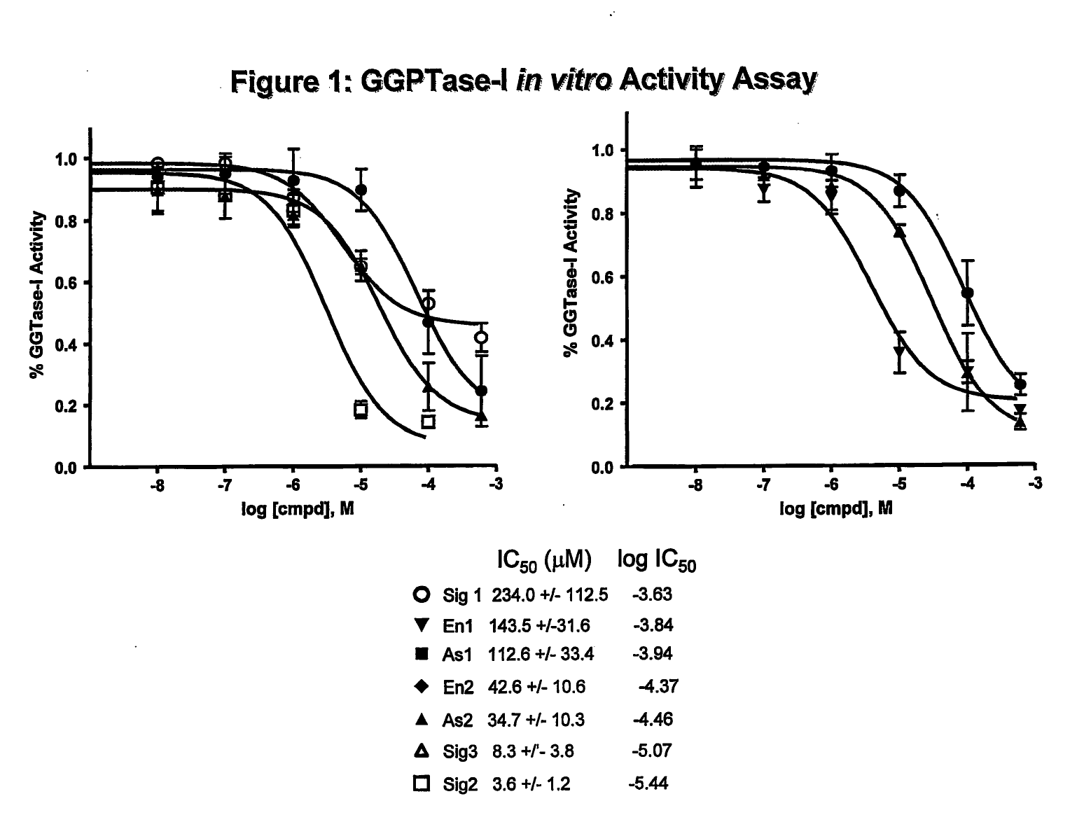

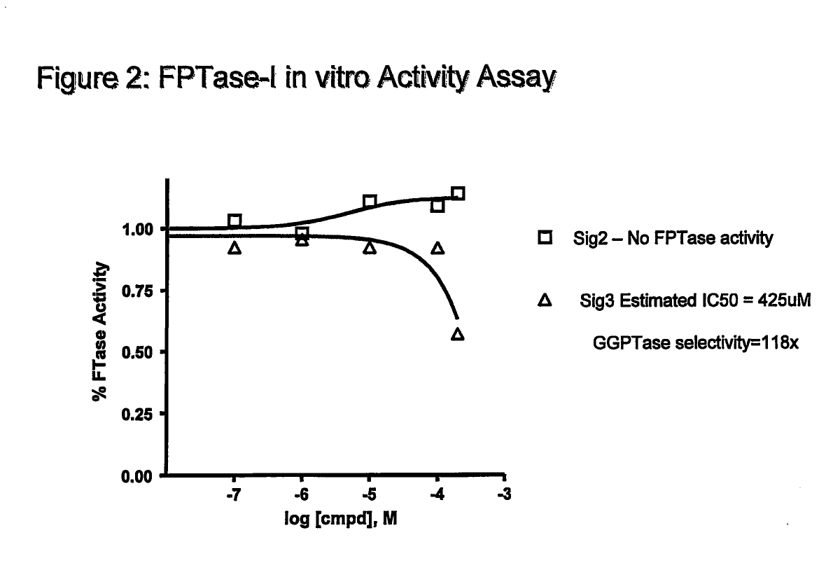

Using the method described above, the GGTase I inhibitory activities of some of the compounds of the present invention were evaluated. Compounds of Table 1 were found to have inhibitory activity and FIG. 1 shows the results of the in vitro assay of the inhibitory activity of these compounds. Mixtures of regioisomers and/or enantiomers are used unless indicated otherwise (for example, the position of substituents on a cyclic or heterocyclic moiety within the backbone of the compounds of the present invention). FIG. 2 shows the selectivity of two of the compounds of the present invention for the inhibition of GGPTase activity compated to FPTase activity. In these studies, the Sig2 and Sig3 compounds of Table 1 showed more than 100-fold selectivity for the inhibition of GGPTase over FPTase.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A pharmaceutical composition useful for inhibiting protein prenylation comprising a pharmaceutically-acceptable excipient and at least one compound having the general structure of Formula I: 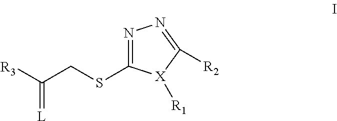
or a pharmaceutically-acceptable salt thereof,
wherein,
X is independently C or N;
L is independently O or CH₂;
R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;
R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-isopropylphenyl, 4-methylphenyl; and,
R₃ is H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, N-(2,5-dichlorophenyl)amide, 3-chloro-N-methylaniline,

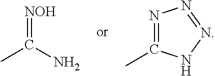

2. A pharmaceutical composition useful for inhibiting protein prenylation comprising a pharmaceutically-acceptable excipient and at least one compound having the general structure of Formula II: 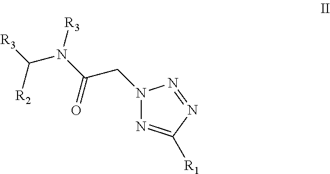
or a pharmaceutically-acceptable salt thereof,
wherein,
R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;
R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-methylphenyl, or 4-isopropylphenyl; and,
Each R₃ is independently H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

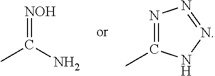

3. A pharmaceutical composition useful for inhibiting protein prenylation comprising a pharmaceutically-acceptable excipient and at least one compound having the general structure of Formula III: 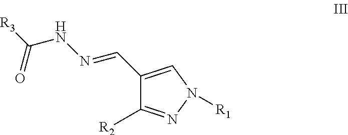
or a pharmaceutically-acceptable salt thereof,
wherein,
R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethylphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl;
R₂ is methyl, pyridine, pyridine-1-oxide, 3-cyanophenyl, 3-aminophenyl, 3-amidinophenyl, 3-dimethylaminophenyl, 2-methylthiazole, thiadiazole, 4-methylthiadiazole, 5-methylisoxazole, 1,3-dimethylpyrazole, 5-methylpyrazole, pyrazine, pyrimidine, 5-methylimidazole, 2-benzylsulfanylpyridine, 6-benzylsulfanylpyridine, acetic acid, N,N-dimethylamine, methylsulfane, ethylbenzene, 1-ethylbenzene, 4-propoxyphenyl, methyl-piperidinyl, 4-methylphenyl, or 4-isopropylphenyl; and,
R₃ is H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

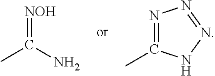

4. A pharmaceutical composition useful for inhibiting protein prenylation comprising a pharmaceutically-acceptable excipient and at least one compound having the general structure of Formula IV: 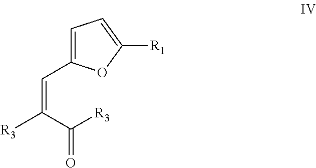
or a pharmaceutically-acceptable salt thereof,
wherein,
R₁ is methyl, ethyl, trifluoroethyl, propyl, benzyl, phenyl, propanol, ethanol, butanol, pyrimidine, 2,4-dimethylpyrimidine, 2-methoxyphenyl, 3-fluorophenyl, 3-chlorophenyl, 3-bromophenyl, 3-methoxyphenyl, 3-chlorophenyl, 3,4-dimethylphenyl, 43-chloropyridazine, 3,4-difluorophenyl, 3,4-dichlorophenyl, 3,5-dichlorophenyl, 3,5-difluorophenyl, 3,4-methylenedioxyphenyl, 4-fluorophenyl, 4-chlorophenyl, 4-bromophenyl, 4-trifluoromethyiphenyl, 4-aminophenyl, 4-nitrophenyl, 4-methylphenyl, 4-methoxyphenyl, 4-chloro-2-methylphenyl, 4-sulfonamidophenyl, or 4-ethylphenoxymethyl; and,
Each R₃ is independently H, NH₂, C(O)N(CH₃)₂, CO₂H, CN, CH₂OH, C(O)NH₂, CSNH₂, C(O)NHOH, C(NH)NH₂, C(O)NHNH₂, C(O)NHCH₃, CH₂OCH₃, C(O)NH-cyclohexyl, CO₂CH₃, 4-aminobenzamide, phenylmethylamine, 1-methyl-2-chlorobenzene, 4-methylbenzamide, 3-chlorobenzamide, 2,5-dichloroaniline, 4-isopropylphenyl, N-(2,5-dichlorophenyl)amide, 3-chloro-N-methylaniline,

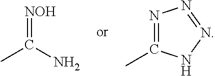

5. A method of treating glaucoma in a mammal comprising administering to the mammal a compound of any one of claim 1.

6. A method of treating macular degeneration in a mammal comprising administering to the mammal a compound of any one of claim 1.

7. A method of treating or preventing the recurrence of psoriasis in a mammal comprising administering to the mammal a compound of any one of claim 1.

8. A method of treating or preventing the recurrence of arthritis in a mammal comprising administering to the mammal a compound of any one of claim 1.

9. A method of treating or preventing the recurrence of a pathology associated with protein prenylation in an organism comprising administering to the organism a compound of any one of claim 1.

10. The method of claim 9, wherein the pathology is selected from the group consisting of cancer, inflammation, multiple sclerosis, restenosis, psoriasis, endometriosis, atherosclerosis, ischemia, myocardial ischemic disorders such as myocardial infarction, high serum cholesterol levels, viral infections including Hepatitis C and HIV, fungal infections, yeast infections, bacterial and protozoan infections, apoptosis, angiogenesis, rheumatoid arthritis, psoriasis, glaucoma, macular degeneration, diabetic retinopathy, disorders related to abnormal angiogenesis, and corneal neovascularization.
